(19) United States
(12) Patent Application Publication
(10) Pub. No.: US 2010/0110242 A1
(43) Pub. Date: May 6, 2010

(54) Anthraquinone dye containing material, composition including the same, camera including the same, and associated methods

(76) Inventors: Shahrokh Motallebi, Los Gatos, CA (US); Eui June Jeong, Seoul (KR); Changmin Lee, Goyang-si (KR); Jae-Hyun Kim, Seongnam-si (KR); Kilsung Lee, Gwacheon-si (KR); Sina Maghsoodi, San Jose, CA (US)

Correspondence Address: LEE & MORSE, P.C., 3141 FAIRVIEW PARK DRIVE, SUITE 500, FALLS CHURCH, VA, 22042, US

(21) Appl. No.: 12/458,174

(22) Filed: Jul. 2, 2009

Related U.S. Application Data
(60) Provisional application No. 61/111,203, filed on Nov. 4, 2008.

(30) Foreign Application Priority Data
May 26, 2009 (KR) 2009-0046036

(51) Int. Cl. H04N 5/335; C07C 69/95; G03F 1/00; G03F 7/20

(52) U.S. Cl. 348/273; 560/51; 430/7; 430/321; 348/E05.091

(57) ABSTRACT

A (meth)acrylate ester includes a (meth)acrylate monomer moiety having an ester oxygen, an anthraquinone moiety having a transmittance spectrum producing red light, and a linking group covalently coupled to the ester oxygen and the anthraquinone moiety, the linking group including phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

### CROSS-REFERENCE TO RELATED APPLICATIONS

This application claims priority from U.S. Provisional Application No. 61/111,203, filed on Nov. 4, 2008 and entitled: “Dye-Containing Methacrylic Polymers in the Composition of Photo Resists for CMOS Sensors,” which is incorporated by reference herein in its entirety for all purposes.

### BACKGROUND OF THE INVENTION

1. Field of the Invention

Embodiments relate to an anthraquinone dye containing material, a composition including the same, a camera including the same, and associated methods.

2. Description of the Related Art

Image sensors based on charge coupled device (CCD) or complementary metal oxide semiconductor (CMOS) technology are widely used in digital imaging devices, e.g., digital still cameras, digital cameras in cell phones, computer web cameras (webcams), etc.

The pixel size of red, green, and blue in a color filter array of an image sensor should be reduced in order to obtain higher-resolution images for a sensor of a given size. In the manufacture of color filters, pigmented color resists have been used. However, pigmented color resists may be heterogeneous, i.e., the pigment may be heterogeneous with respect to the resist matrix. Accordingly, pigmented color resists may not provide sufficient lithographic resolution, making the manufacture of high-resolution image sensors difficult. Further, pigmented color resists may leave behind residues after patterning of the color filter.

Dye-based color resists may be used instead of pigmented color resists. Such dye-based color resists may provided enhanced homogeneity and may leave less residue than pigmented color resists. However, dye-based color resists may not afford desired levels of thermal stability, light stability, and chemical stability.

### SUMMARY OF THE INVENTION

Embodiments are therefore directed to an anthraquinone dye containing material, a composition including the same, a camera including the same, and associated methods, which substantially overcome one or more of the problems due to the limitations and disadvantages of the related art.

Features and advantages of the present invention may be realized by providing a (meth)acrylate ester, including a (meth)acrylate monomer moiety having an ester oxygen, an anthraquinone moiety having a transmittance spectrum producing red light and a linking group covalently coupled to the ester oxygen and the anthraquinone moiety, wherein the linking group includes phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

The linking group may be derived from an amino alcohol.

The anthraquinone moiety may have the linking group at the 1-position only.

Features and advantages of the present invention may also be realized by providing a method of synthesizing a (meth)acrylate ester, the method including providing an anthraquinone compound having a reactive group attached to a ring of the anthraquinone ring, reacting a linking group with the reactive group such that the linking group becomes covalently bound to the ring of the anthraquinone, and reacting a (meth)acrylic acid-derived compound with the linking group such that the linking group becomes covalently bound to an oxygen of the (meth)acrylic acid-derived compound. The linking group may include phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

The linking group may be an amino alcohol.

The anthraquinone moiety may have the linking group at the 1-position only.

The (meth)acrylic acid-derived compound may be a (meth)acrylic acid anhydride.

Features and advantages of the present invention may also be realized by providing a method of manufacturing a camera, the method including fabricating a color filter, and mounting the color filter proximate to a sensor array, the color filter including a red filter region. Forming the red filter region may include patterning a red color photoresist, and the red color photoresist may include a polymer having a backbone at least a portion of which corresponds to a (meth)acrylate that includes an anthraquinone moiety having a transmittance spectrum producing red light, and a linking group covalently coupled to the anthraquinone moiety and an ester oxygen of the (meth)acrylate, the linking group including phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

Patterning the red color photoresist may include exposing the red color photoresist to i-line radiation.

The red color photoresist may be a negative resist, the polymer may include at least one functional group that is polymerizable with another component of the red color photoresist by exposure to UV light, and the polymer backbone may include at least one functional group that is reactive when placed in contact with aqueous alkaline developer.

The red color photoresist may include the polymer, a crosslinker, and an initiator that is reactive to UV light.

The crosslinker may include an acrylate oligomer.

The acrylate oligomer may include a polyol (meth)acrylate ester.

Patterning the red color photoresist may include forming a red color pixel having a width of about 2 μm or less.

Patterning the red color photoresist may include forming a red color pixel having a width of about 1.4 μm or less.

Features and advantages of the present invention may also be realized by providing a camera, including a sensor array, and a color filter proximate to the sensor array, the color filter including a red filter region. The red filter region may include a red color photoresist, and the red color photoresist may include a polymer having a backbone at least a portion of which corresponds to a (meth)acrylate that includes an anthraquinone moiety having a transmittance spectrum producing red light, and a linking group covalently coupled to the anthraquinone moiety and an ester oxygen of the (meth)acrylate, the linking group including phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

The red filter region may include a red color pixel having a width of about 2 μm or less.

The red filter region may include a red color pixel having a width of about 1.4 μm or less.

### BRIEF DESCRIPTION OF THE DRAWINGS

The above and other features and advantages will become more apparent to those of ordinary skill in the art by describing in detail example embodiments with reference to the attached drawings, in which:

FIG. 1 illustrates Formulae II-VI representing (meth)acrylic dye monomers according to embodiments;

FIGS. 2A-2B illustrate the chemical structure and transmittance spectrum of Solvent Red 111;

FIGS. 3A-3C illustrate syntheses of (meth)acrylic dye-containing monomers according to embodiments;

FIGS. 4A-4F illustrate transmittance spectra of methacrylic dye monomers according to embodiments;

FIGS. 5A-5D illustrate CD-SEM images of 1.4 μm patterns formed using a photoresist according to an embodiment; and

FIG. 6 illustrates a schematic diagram of a camera according to an embodiment.

### DETAILED DESCRIPTION OF THE INVENTION

Korean Patent Application No. 2009-0046036, filed on May 26, 2009, in the Korean Intellectual Property Office, and entitled: “(Meth)acrylate Compound, Photoresist and Image Sensor Including the Same,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art. In the figures, the dimensions of layers and regions may be exaggerated for clarity of illustration. Like reference numerals refer to like elements throughout.

As used herein, the expressions “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C” and “A, B, and/or C” includes the following meanings: A alone; B alone; C alone; both A and B together; both A and C together; both B and C together; and all three of A, B, and C together. Further, these expressions are open-ended, unless expressly designated to the contrary by their combination with the term “consisting of:” For example, the expression “at least one of A, B, and C” may also include an n^{th} member, where n is greater than 3, whereas the expression “at least one selected from the group consisting of A, B, and C” does not.

As used herein, the expression “or” is not an “exclusive or” unless it is used in conjunction with the term “either.” For example, the expression “A, B, or C” includes A alone; B alone; C alone; both A and B together; both A and C together; both B and C together; and all three of A, B, and C together, whereas the expression “either A, B, or C” means one of A alone, B alone, and C alone, and does not mean any of both A and B together; both A and C together; both B and C together; and all three of A, B, and C together.

As used herein, the terms “a” and “an” are open terms that may be used in conjunction with singular items or with plural items. For example, the term “a solvent” may represent a single solvent or multiple solvents in combination.

As used herein, molecular weights of polymeric materials are weight average molecular weights, unless otherwise indicated.

As used herein, the term “(meth)acrylate” refers to both acrylate and methacrylate. Thus, for example, the term ethyl(meth)acrylate refers to both ethyl acrylate and ethyl methacrylate. Further, the term “acrylate” is generic to both acrylate and methacrylate, unless specified otherwise. Thus, ethyl acrylate and ethyl methacrylate are both acrylates.

Embodiments relate to an anthraquinone dye containing material, a composition including the same, a camera including the same, and associated methods. The dye-containing methacrylic material may be a polymer represented by Formula I below:

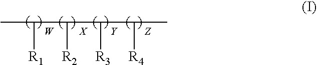

In Formula I, the unit R₁ may be derived from a monomeric unit that imparts red color to the polymer. The monomeric unit R₁ may include an acrylic or methacrylic moiety (the acrylic or methacrylic moiety being generically referred to as a “(meth)acrylic moiety”) and a dye moiety covalently bound thereto. The dye moiety may be an anthraquinone moiety.

In Formula I, the units R₂ and R₃ may be derived from olefinic monomers and may be different from one another. The unit R₄ may be an olefinic monomer having a carboxyl group.

The dye-containing methacrylic material represented by Formula I may be a random copolymer. In Formula I, W+X+Y+Z=1. The fraction W of the unit R₁ may be about 10 mole percent (“mol. %”) to about 70 mol. % of the polymer, preferably about 20 mol. % to about 50 mol. %. The fraction X of the unit R₂ may be about 0 mol. % to about 50 mol. % of the polymer (it will be understood that a fraction of 0 mol. % indicates that the fraction may be omitted). In an implementation, the fraction X of the unit R₂ is from greater than 0 mol. % to about 50 mol. % of the polymer, i.e., the fraction is present in the polymer). The fraction Y of the unit R₃ may be about 10 mol. % to about 50 mol. % of the polymer. The fraction Z of the unit R₄ may be about 5 mol. % to about 50 mol. % of the polymer, preferably about 15 mol. % to about 30 mol. %. As discussed above, W+X+Y+Z=1. Accordingly, the sum of the mol. % of the fractions W, X, Y, and Z totals 100 mol. %. The dye-containing (meth)acrylic polymer may be formed by polymerizing a (meth)acrylic dye-containing monomer according to an embodiment with moieties corresponding to units R₂-R₄ described above.

The dye-containing (meth)acrylic polymer represented by Formula I may have a molecular weight of about 2,000 to about 50,000, preferably about 4,000 to about 20,000.

The unit R₁ may include a linking group between the dye moiety and the (meth)acrylic functional group. The linking group may serve to enable polymerization by reducing steric hindrance by positioning the dye moiety apart from the (meth)acrylic functional group. The linking group may be, e.g., an amino alcohol, i.e., a compound having an amine functional group and a hydroxyl group. The amino alcohol may be an alkyl or aryl amino alcohol. The linking group may include phenyl, naphthyl, a linear alkyl group having from 2 to about 10 carbons, a branched alkyl group having from 3 to about 10 carbons, a cycloalkyl group having from about 3 to about 20 carbons, or a substituted aromatic group. In another implementation, the linking group may include phenyl, naphthyl, a linear alkyl group having from 3 to about 10 carbons, a branched alkyl group having from 4 to about 10 carbons, a cycloalkyl group having from about 3 to about 20 carbons, or a substituted aromatic group.

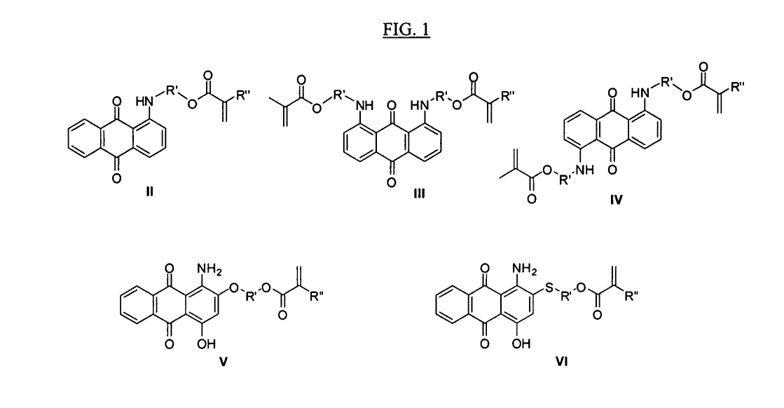

Particular examples of the unit R₁ are represented by Formulae II through VI shown in FIG. 1 and reproduced below:

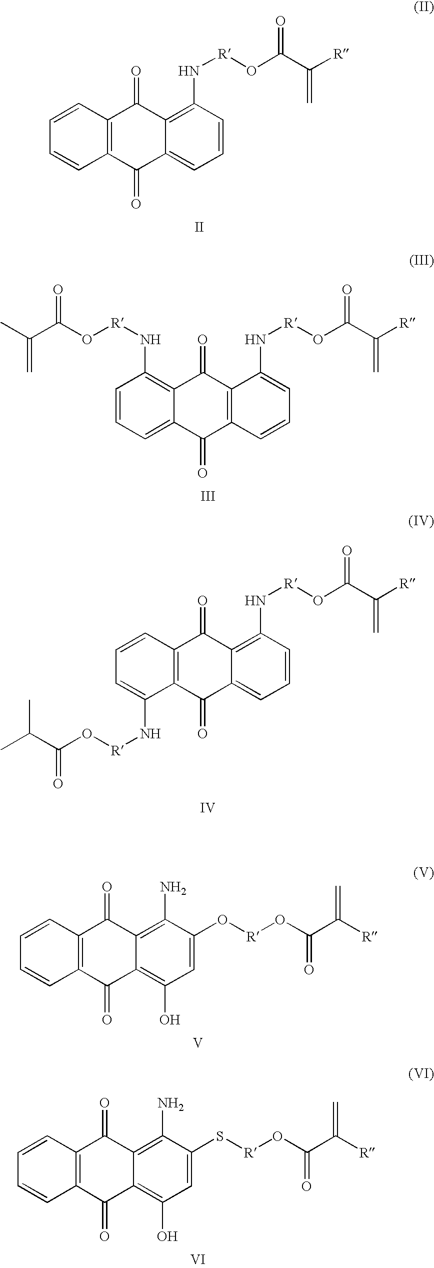

In Formulae II through VI, R′ may be, e.g., phenyl, naphthyl, a linear alkyl group having from 1 to about 10 carbons, a branched alkyl group having from 3 to about 10 carbons, a cycloalkyl group having from about 3 to about 20 carbons, or a substituted aromatic group. In an implementation, in Formula II through VI, R′ may be, e.g., phenyl, naphthyl, a linear alkyl group having from 2 to about 10 carbons, a branched alkyl group having from 3 to about 10 carbons, a cycloalkyl group having from about 3 to about 20 carbons, or a substituted aromatic group. In another implementation, in Formula II through VI, R′ may be, e.g., phenyl, naphthyl, a linear alkyl group having from 3 to about 10 carbons, a branched alkyl group having from 4 to about 10 carbons, a cycloalkyl group having from about 3 to about 20 carbons, or a substituted aromatic group.

Where more than one group R′ is present, e.g., in Formula II, each R′ may be independently selected, i.e., the all R′ groups in the polymer need not be the same.

In Formulae II through VI, R″ may be, e.g., hydrogen or methyl. Where more than one group R″ is present, each R″ may be independently selected.

The units R₂ and R₃ may provide solubility to the polymeric structure. The units R₂ and R₃ may be olefinic polymerizable monomers and may be different from one another.

In an implementation, the units R₂ and R₃ may each be derived from esters of (meth)acrylic acids, i.e., esters of acrylic acids and esters of methacrylic acids. For example, R₂ and R₃ may be allyl(meth)acrylate, methyl(meth)acrylate, ethyl(meth)acrylate, n-propyl(meth)acrylate, isopropyl(meth)acrylate, butyl(meth)acrylate, tetrahydrofurfuryl(meth)acrylate, cyclohexyl(meth)acrylate, isobornyl(meth)acrylate, n-hexyl(meth)acrylate, n-octyl(meth)acrylate, isooctyl(meth)acrylate, 2-ethylhexyl(meth)acrylate, lauryl(meth)acrylate, glycidyl(meth)acrylate, stearyl(meth)acrylate, phenyl(meth)acrylate, benzyl(meth)acrylate, 2-hydroxyethyl(meth)acrylate, 2-hydroxybutyl(meth)acrylate, 2-aminoethyl(meth)acrylate, or 2-dimethylaminoethyl(meth)acrylate.

In an implementation, the units R₂ and R₃ may each be derived from styrenes. For example, R₂ and R₃ may be derived from styrene, α-methylstyrene, vinyltoluene, or vinylbenzyl methyl ether.

In an implementation, the units R₂ and R₃ may each be derived from carboxylic acid vinyl esters. For example, R₂ and R₃ may be derived from vinyl acetate and vinylbenzoate, vinyl cyanide compounds such as acrylonitrile and methacrylonitrile, or unsaturated amides such as acrylamide and methacrylamide.

The unit R₄ may provide solubility in an aqueous alkaline solution, e.g., a photo resist developer solution. The unit R₄ may be an olefinic polymerizable compound having a carboxylic acid group. The unit R₄ may be, e.g., (meth)acrylic acid (i.e., acrylic acid or methacrylic acid), maleic acid, itaconic acid, or fumaric acid.

The dye-containing methacrylic polymer may be used in a photoresist composition sensitive to radiation at, e.g., a 365 nm (“i-line”) wavelength. In an implementation, the dye-containing methacrylic polymer may include an acid moiety in the polymer, such that the polymer can be used in a photoresist composition that exhibits negative resist characteristics, i.e., in a photoresist composition that polymerizes in regions exposed to radiation, and where unexposed regions are removed upon developing. The developer may be an alkaline developer such as an aqueous tetramethyl ammonium hydroxide (“TMAH”) solution. In another implementation, the photoresist composition may be a positive resist.

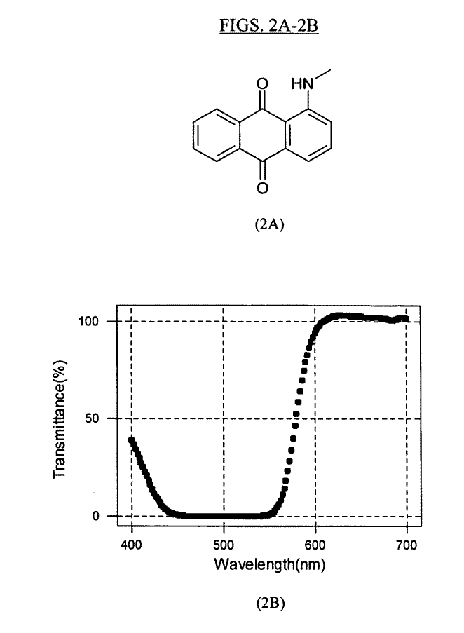

In an embodiment, the (meth)acrylic dye-containing monomers according to embodiments may be formed by modifying a dye moiety with the linking group, and then bonding the modified dye moiety to a (meth)acrylic functional group. The dye moiety may be an anthraquinone moiety similar to Solvent Red 111 shown in FIG. 2A. The transmittance spectrum of Solvent Red 111 is shown in FIG. 2B. As compared to other red dyes such as Solvent Red 119, 122, 124, 160 and 179, the transmittance spectrum of Solvent Red 111 may be best suited for use in the fabrication of color filters for CMOS sensors. As discussed in detail below, (meth)acrylic dye-containing monomers according to embodiments may have transmittance spectra similar to that of Solvent Red 111.

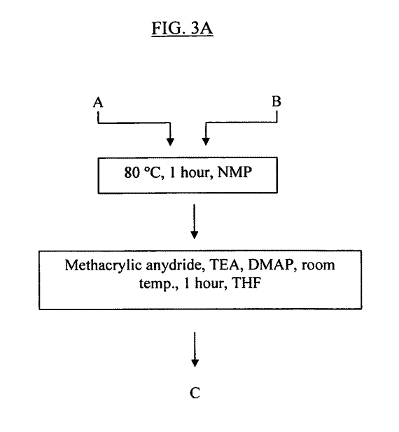

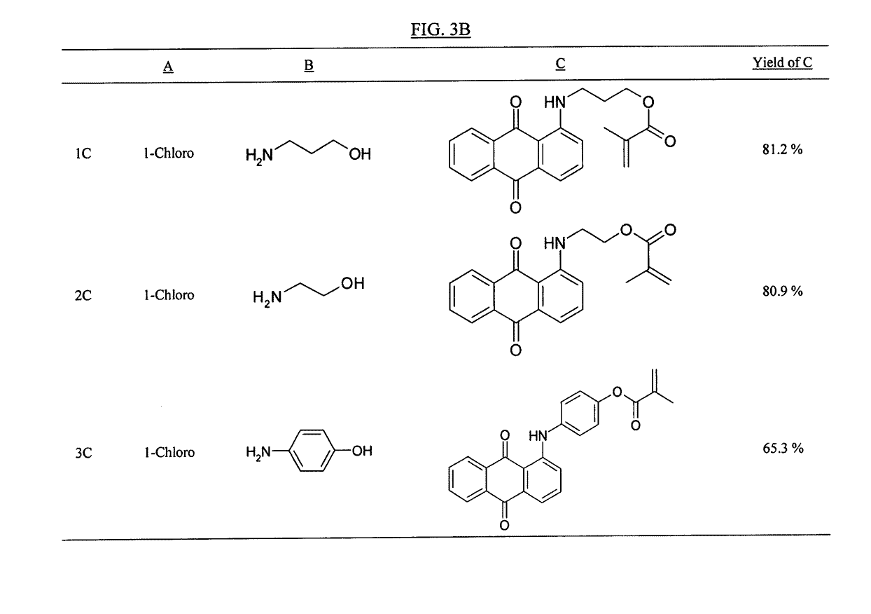

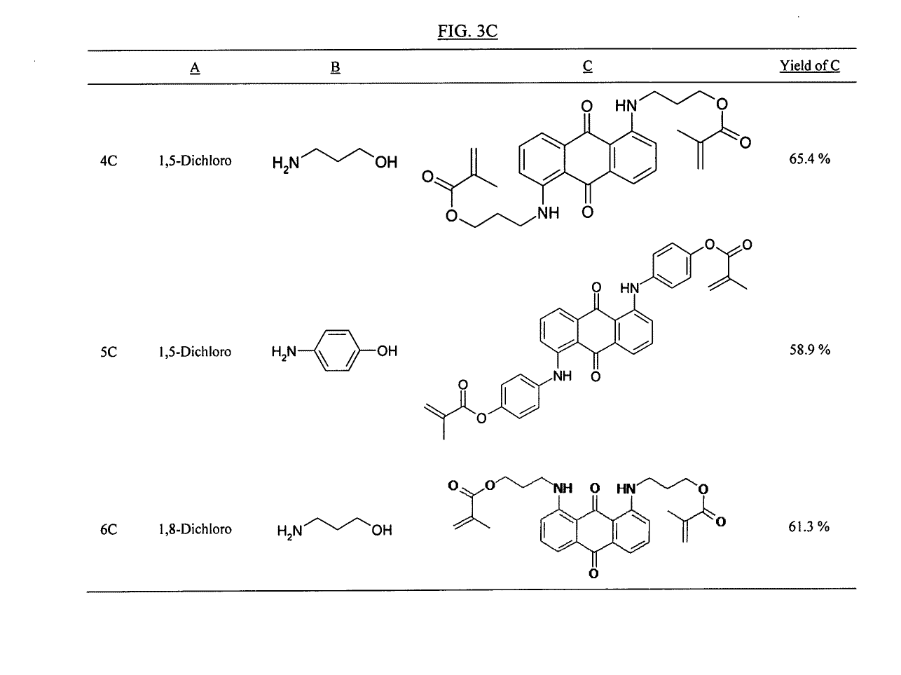

FIGS. 3A-3C illustrate syntheses of (meth)acrylic dye-containing monomers according to embodiments. Referring to FIG. 3A, components A and B may be reacted together, and subsequently combined with a (meth)acrylic functional group such as methacrylic anhydride to form (meth)acrylic dye-containing monomer C. In an implementation, the reaction of components A and B may be performed at a temperature of about 80° C. for 1 hour in a solvent solution of, e.g., N-methyl pyrrolidone (“NMP”). In an implementation, the reaction product of components A and B may then be combined with an amine base, e.g., triethylamine (“TEA”), and dimethylaminopyridine (“DMAP”) in an aprotic organic solvent such as tetrahydrofuran (“THF”). The mixture may be allowed to react at room temperature for a period of, e.g., 1 hour.

Examples of dye component A, linking component B, and the resultant (meth)acrylic dye-containing monomer C are shown in the table spanning FIGS. 3B and 3C. In FIGS. 3B and 3C, component A refers to the substitution of anthraquinone, e.g., “1-chloro” refers to 1-chloroanthraquinone. In the dye-containing (meth)acrylic polymer represented by Formula I, the unit R₁ corresponds to monomer C.

Referring to FIGS. 3B and 3C, dye-containing (meth)acrylic monomers 1C, 2C and 3C correspond to Formula II in FIG. 1, dye-containing (meth)acrylic monomers 4C and 5C correspond to Formula IV in FIG. 1, and dye-containing (meth)acrylic monomer 6C corresponds to Formula III in FIG. 1. Details regarding example dye-containing (meth)acrylic monomers 1C through 6C are given below.

### Formula II

Example dye-containing (meth)acrylic monomer 1C:

2-Methyl-acrylic acid-3-(9,10-dioxo-9,10-dihydroanthracene-1-ylamino)propyl ester, ¹H NMR (300 MHz, CDCl₃) δ 9.81 (br, 1H), 8.33-8.23 (m, 3H), 7.84-7.73 (m, 4H), 6.15 (t, 1H), 5.58 (m, 1H), 4.35 (t, 2H), 3.48 (q, 2H), 2.15 (m, 2H), 1.97 (t, 3H)

Example dye-containing (meth)acrylic monomer 2C:

2-Methyl-acrylic acid-2-(9,10-dioxo-9,10-dihydroanthracene-1-ylamino)ethyl ester, ¹H NMR (300 MHz, CDCl₃) δ 9.72 (br, 1H), 8.37-8.23 (m, 3H), 7.84-7.52 (m, 4H), 6.10 (t, 1H), 5.47 (m, 1H), 4.18 (t, 2H), 3.42 (q, 2H), 1.96 (t, 3H)

Example dye-containing (meth)acrylic monomer 3C:

2-Methyl-acrylic acid-4-(9,10-dioxo-9,10-dihydroanthracene-1-ylamino)phenyl ester, ¹H NMR (300 MHz, CDCl₃) δ 11.34 (br, 1H), 8.35-8.26 (m, 4H), 7.86-7.49 (m, 3H), 7.36-7.18 (m, 4H), 6.38 (s, 1H), 5.79 (s, 1H), 1.58 (s, 3H)

### Formula IV

Example dye-containing (meth)acrylic monomer 4C:

2-Methyl-acrylic acid-3-{5-[3-(2-methyl-acryloyloxy)-propylamino]-9,10-dioxo-9,10-dihydroanthracene-1-ylamino}propyl ester, ¹H NMR (300 MHz, CDCl₃) δ 9.77 (s, 2H), 7.58-7.50 (m, 3H), 6.99-6.97 (m, 3H), 6.14 (t, 2H), 5.59 (m, 2H), 4.35 (q, 4H), 3.12 (m, 4H), 1.99 (s, 6H)

Example dye-containing (meth)acrylic monomer 5C:

2-Methyl-acrylic acid-4-{5-[4-(2-methyl-acryloyloxy)-phenylamino]-9,10-dioxo-9,10-dihydroanthracene-1-ylamino}phenyl ester, ¹H NMR (300 MHz, CDCl₃) δ 11.17 (br, 2H), 8.29-8.26 (m, 3H), 7.80 (m, 3H), 7.75-7.66 (m, 8H), 6.38 (s, 2H), 5.79 (s, 2H), 1.64 (br, 6H)

### Formula III

Example dye-containing (meth)acrylic monomer 6C:

2-Methyl-acrylic acid-3-{8-[3-(2-methyl-acryloyloxy)-propylamino]-9,10-dioxo-9,10-dihydroanthracene-1-ylamino}propyl ester, ¹H NMR (300 MHz, CDCl₃) δ 9.66 (br, 2H), 7.55 (d, 2H), 7.48 (t, 2H), 7.00 (d, 2H), 6.15 (s, 2H), 5.59 (s, 2H), 4.35 (t, 4H), 3.47 (q, 4H), 2.14 (m, 4H), 1.97 (s, 6H)

The reaction scheme described above in connection with FIG. 3A may provide good yields of (meth)acrylic dye-containing monomer C, e.g., yields of 50% to 80% or more based on the amount of the dye component A. Further, the reaction scheme based on a chloro-substituted anthraquinone may be more effective that, e.g., an amide coupling scheme starting from an amine-substituted anthraquinone such as Solvent Red 111, as reactions with the amine-substituted anthraquinone may be impeded by hydrogen bonding between the carbonyl and the amine hydrogen.

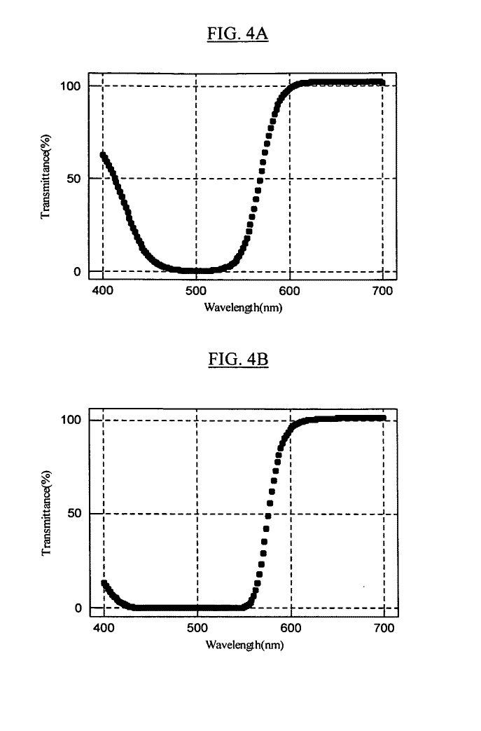

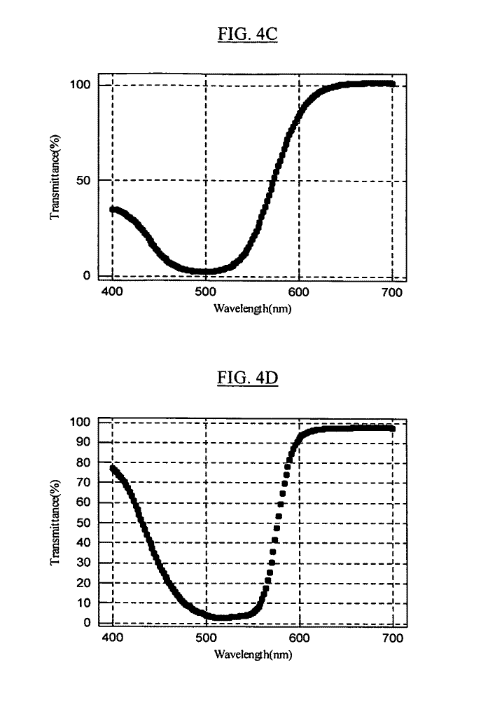

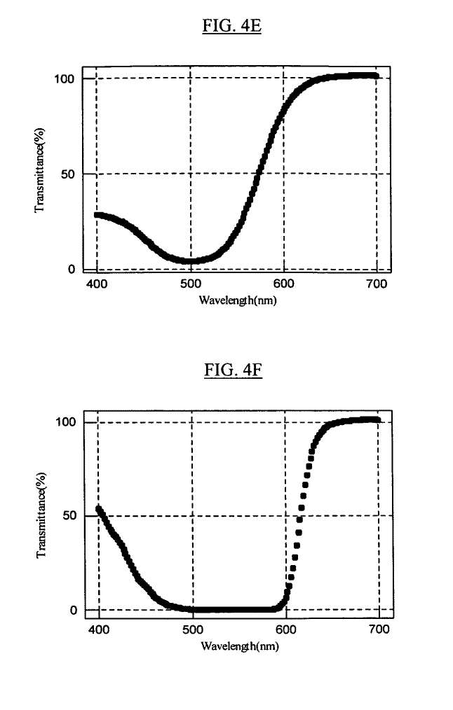

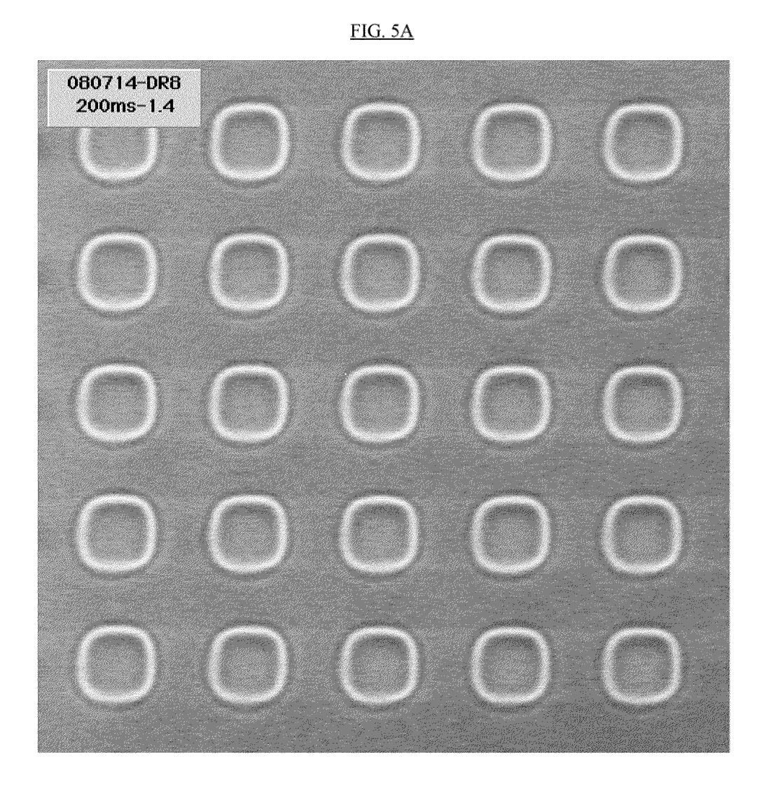

The (meth)acrylic dye-containing monomers 1C-6C set forth in FIGS. 3B-3C may exhibit transmittance spectra, shown in FIGS. 4A-4F, having ultraviolet-visible (“UV-VIS”) region (about 400 nm to about 700 nm) transmittance similar to that of Solvent Red 111, i.e., a red color. In some cases, the transmittance minima and/or maxima may be shifted relative to Solvent Red 111.

As discussed above, thermal stability of the monomer is an important consideration in applications of the monomers to form photoresists for color filters. For each of the (meth)acrylic dye-containing monomers 1C-6C, the thermal stability of was measured by thermal gravimetric analysis (“TGA”). The measurements demonstrate that the 5% weight loss-temperatures of the (meth)acrylic dye-containing monomers may vary considerably based on the type and number of the linking moieties (component B).

With particular respect to the (meth)acrylic dye-containing monomers 1C-6C, in the case of the propyl linking moiety, the 5 wt % loss temperature of the (meth)acrylic dye-containing monomers with the a single propyl linking moiety (monomer 1C) and two propyl linking moieties (monomer 6C) were observed to be 367.53° C. and 266.06° C., respectively. The 5 wt % loss temperature of the (meth)acrylic dye-containing monomers with a single phenyl linking moiety (monomer 3C) and the two phenyl linking moieties (monomer 5C) were observed to be 211.91° C. and 270.62° C., respectively. For reference, the 5 wt % loss temperature of Solvent Red 111 was 247.87° C.

### Example Syntheses of Dye-Containing (meth)acrylic Monomers Represented by Formulae II, III and IV

An example method suitable for the synthesis of monomers represented by Formulae II, III, and IV will now be described. First, a solution of a chloroanthraquinone (e.g., mono- or di-chloro substituted anthraquinone) (1 mole equivalent), a corresponding amino alcohol (3.0 equiv.) in NMP may be heated to a temperature of about 80° C. for a period of about 1 hour to about 5 hours. An atmosphere of an inert gas such as nitrogen may be used to blanket the solution. After the 1-5 hr. period, the resulting solution may be cooled to room temperature and precipitated in water. After filtration and rinsing with water, the resulting solid may be crystallized in a suitable solvent such as acetonitrile to provide an intermediate hydroxyl alkylamino anthraquinone (dye-linking component (A-B)) compound.

Subsequently, a solution of the intermediate hydroxyl alkylamino anthraquinone compound (1.0 equiv.) and a (meth)acrylic anhydride (1.2 equiv.) in THF may be prepared, and a solution of DMAP (0.2 equiv.) and TEA (1.2 equiv.) in THF may be added drop-wise thereto over a period of, e.g., 60 min., at room temperature. After adding the DMAP/TEA solution, the reaction mixture may be stirred until the reaction completes. The progress of the reaction may be monitored using any suitable technique such as thin film chromatography, etc. After the reaction is complete, the reaction mixture may be neutralized using, e.g., acetic acid. The reaction mixture may then be precipitated in water, filtered, washed with water and recrystallized, e.g., in acetonitrile, to produce the (meth)acrylic dye-containing monomer C.

### Polymerization Examples for Dye-Containing (Meth)Acrylic Monomers Represented by Formulae II, III and IV

A polymer may be formed by polymerizing three kinds of monomers such as a (meth)acrylic dye-containing monomer, benzyl methacrylate (“BzMA”) and methacrylic acid (“MAA”). Polymerization may be performed in, e.g., THF.

For example, a terpolymer prepared using monomer 1C produced a polymer having a weight average molecular weight (“Mw”) of 20.0 kD. Corresponding terpolymers produced using monomers 3C, 4C and 5C in place of monomer 1C had respective average molecular weights of 5.6 kD, 20.6 kD, and 16.0 kD, and respective polymerization yields of 62%, 58%, and 60%.

The Mw of the polymers were dependent on the amount of initiator. In particular, as the amount of initiator was increased, the Mw decreased, presumably because the starting points of the polymerization were increased in a limited environment. The Mw of the polymer can thus be controlled by controlling the amount of initiator. In an implementation, the amount of initiator may be about 7 wt % with respect to the total weight of monomers.

In an example synthesis, a THF solution containing each of the three kinds of monomers and a radical initiator, e.g., AIBN, may be flushed with nitrogen for 30 min. and then heated to reflux under a nitrogen atmosphere. The solution may be stirred at the reflux temperature for a period sufficient for the monomers to react, e.g., 10 hours or more. The solution may then be cooled to room temperature and precipitated in hexanes and filters. The filtered solids may be rinsed in hexanes and then dried under vacuum to produce the desired terpolymer.

It will be appreciated that the particular solvent used for the polymerization reaction may depend on the nature of the dye-containing (meth)acrylic monomer. In this regard, the solubility of the monomer and the resulting polymerization product may be important in determining the choice of solvent and controlling the yield of the polymerization reaction. The (meth)acrylic dye-containing monomer (1C) may be largely insoluble in a solvent such as propylene glycol monomethyl ether acetate (“PGMEA”). THF, NMP and dimethyl formamide (“DMF”) may better dissolve the monomers than solvents such as toluene, acetonitrile, ethyl acetate, dichloromethane, n-hexane, and methyl alcohol. Example polymerizations performed in THF, NMP and DMF produced yields of 67%, 34%, and 31%, respectively.

The reaction mixture was stirred to polymerize for 6 hr and then cooled to room temperature. The resultant cooled reaction mixture was added into an excess of n-hexane to form a precipitate. The precipitate was filtered and dried to obtain the corresponding dye-containing (meth)acrylic polymer. The molecular weight of the dyed polymer was characterized by gel permeation chromatography.

### Example Syntheses of Dye-Containing (Meth)Acrylic Monomers Represented by Formula V

### Synthesis of 1-amino-4-hydroxy-2-(2-hydroxyethoxy)anthracene-9,10-dione Starting Material

A solution of Disperse red 60 (200 g), ethylene glycol (800 g), sodium hydroxide (17 g) and NMP (500 ml) was heated under reflux for 2 hr. under nitrogen atmosphere. The mixture was then cooled down to room temperature and precipitated in 1% solution of sulfuric acid in water. The precipitate was then filtered, rinsed with water and dried under vacuum at 45° C.

### Synthesis of 2-(1-amino-4-hydroxy-9,10-dioxo-9,10-dihydroanthracen-2-yloxy)ethyl methacrylate (Formula V in FIG. 1, wherein R′═—CH₂CH₂— and R″═—CH₃)

A solution of 1-amino-4-hydroxy-2-(2-hydroxyethoxy)anthracene-9,10-dione (30 g) and methacrylic anhydride (22 g) in THF (300 g) was prepared, to which a solution of triethylamine (15 g) and DMAP (2.5 g) in THF (75 g) was added dropwise over a period of 60 min. at room temperature. The mixture was stirred at room temperature until the completion was confirmed by TLC. Acetic acid was then added to neutralize the reaction mixture, and then the thus obtained mixture was precipitated in water, filtered, washed with deionized water, and dried under vacuum at 40° C.

### Synthesis of 1-amino-4-hydroxy-2-(6-hydroxyhexyloxy)anthracene-9,10-dione Starting Material

A solution of Disperse red 60 (66.2 g), ethylene glycol (141.6 g), potassium carbonate (27.6 g) and dimethylformamide (“DMF”) (300 g) was heated under reflux for 16 hr. under nitrogen atmosphere. The mixture was then cooled down to 70° C. and ethanol (360 g) was added to the solution. The mixture was then cooled down to room temperature. Acetic acid (24 g) was added to the resulting solution and the mixture was stirred for 10 min. The solution was then filtered and the solid rinsed with ethanol. The solid was then dried under vacuum at 40° C. and then recrystallized in acetonitrile.

### Synthesis of 6-(1-amino-4-hydroxy-9,10-dioxo-9,10-dihydroanthracen-2-yloxy)hexyl methacrylate

A solution of 1-amino-4-hydroxy-2-(6-hydroxyhexyloxy)anthracene-9,10-dione (45.42 g) and methacrylic anhydride (23.7 g) in THF (450 g) was prepared, and a solution of triethylamine (16.8 g) and DMAP (3.1 g) in THF (150 g) was added thereto over a period of 60 min. at room temperature. The mixture was stirred at room temperature until the reaction completion was confirmed by thin layer chromatography. Acetic acid was then added to neutralize the reaction mixture, and the resultant mixture was precipitated in water, filtered, washed with deionized water, and dried under vacuum at 40° C.

Thioxy-type dye-containing (meth)acrylic monomers represented by Formula VI may be prepared in similar fashion to the dye-containing (meth)acrylic monomers represented by Formula V.

### Polymerization Example for Dye-Containing (Meth)Acrylic Monomer Represented by Formula V

A solution of 2-(1-amino-4-hydroxy-9,10-dioxo-9,10-dihydroanthracen-2-yloxy)ethyl methacrylate (3.0 g), methylmethacrylate (4.0 g), methacrylic acid (3.0 g) and radical initiator azobisisobutyronitrile (“AIBN”) (0.5 g) in THF (50 g) was flushed with nitrogen for 30 min. and then heated to 66° C. and allowed to reflux under nitrogen atmosphere. The solution was stirred at this temperature for 16 hr. The solution was then cooled to room temperature and precipitated in 500 g of hexanes, filtered, and then rinsed with 200 g hexanes. The resultant solid was then dried overnight in a vacuum oven at 35° C. to produce a dye-containing (meth)acrylic polymer.

### Photoresist Formulation Example for Dye-Containing (Meth)Acrylic Polymers

Respective dye-containing (meth)acrylic polymers (0.33 g), a base polymer (2.68 g), dipentaerythritol hexaacrylate (“DPHA”) (0.90 g) and a triazine-type photoinitiator (0.14 g) were added in co-solvent (8.53 g) of propylene glycol monomethyl ether acetate (“PGMEA”), ethyl 3-ethoxy propionate, and cyclohexanone. The resulting solution was stirred for 1 hr. to complete dissolution.

### Photopatterning Tests for Photoresist Formulations

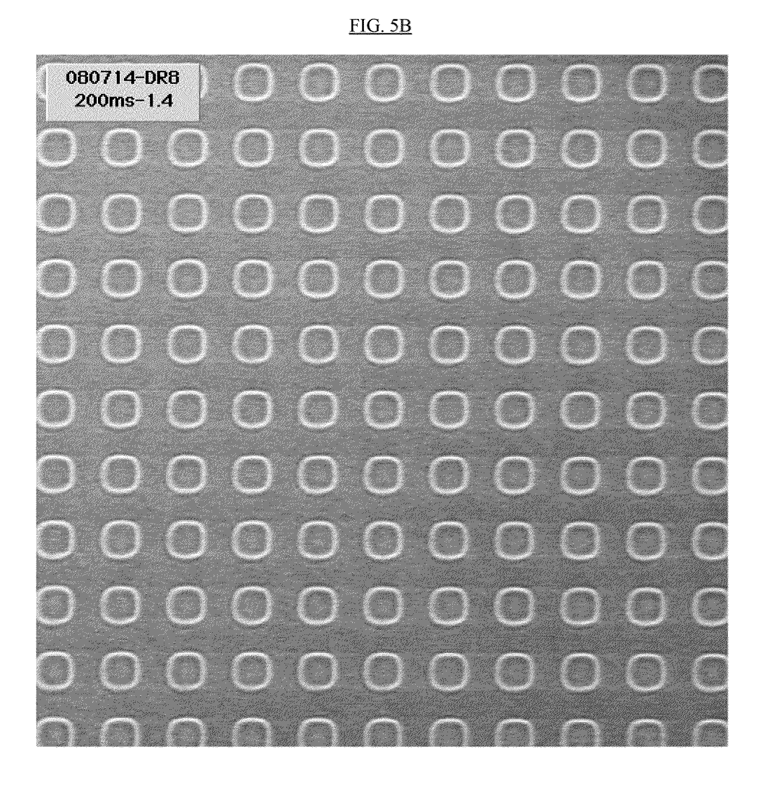

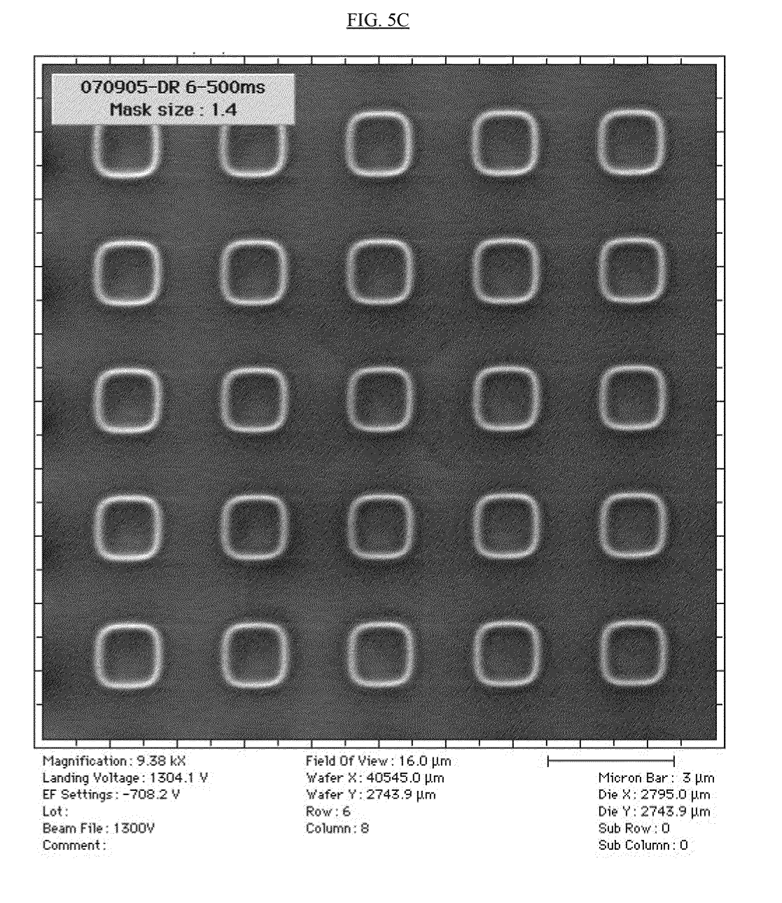

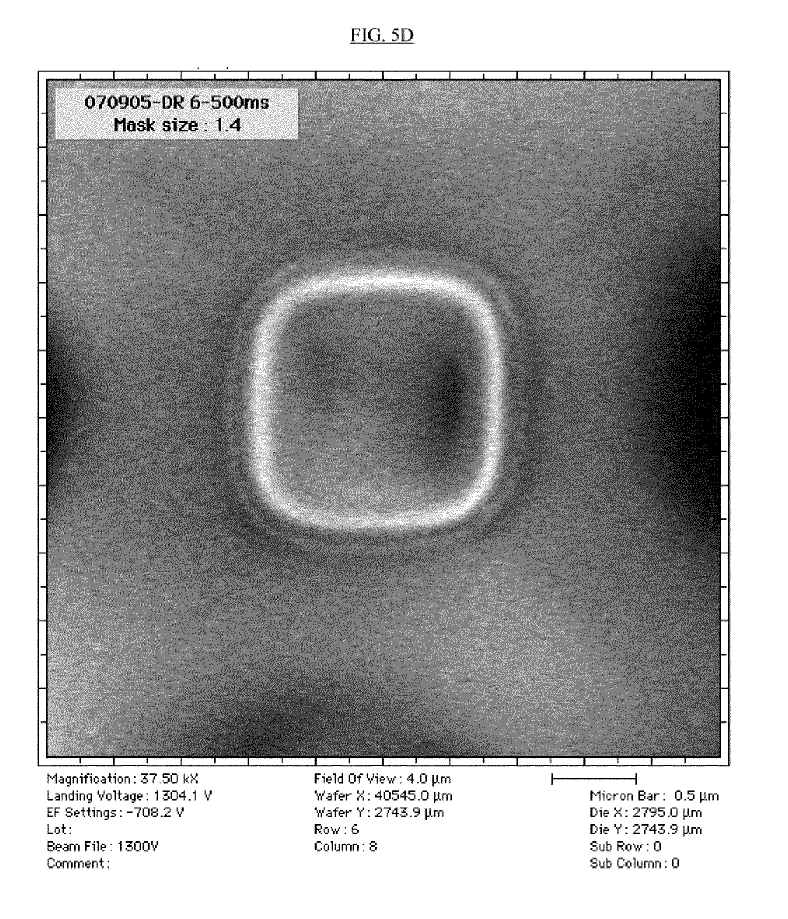

A red anthraquinone dye-containing (meth)acrylic polymer-based photoresist formulated as described directly above was spin-coated to give a 6000 Angstrom thickness on a 200 mm silicon wafer. The coated wafer was baked at 100° C. for 180 s. (seconds), exposed at i-line wavelength (365 nm) for 100-1000 ms., developed with 0.2% aqueous TMAH 120 s., and then baked at 200° C. for 300 s. The resulting patterns were observed by CD SEM. CD-SEM images of the resulting 1.4 patterns are shown in FIGS. 5A-5B.

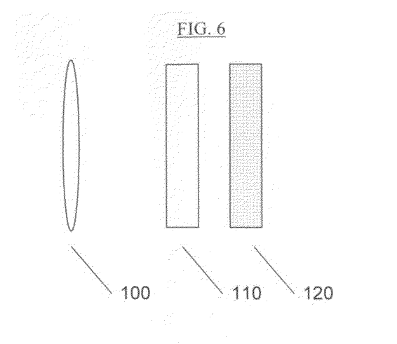

FIG. 6 illustrates a schematic diagram of a camera according to an embodiment. The camera may include a sensor array 120 and a color filter 110 proximate to the sensor array 120. The camera may further include an optically transparent cover or lens 100. Light may enter the lens 100 and pass through the color filter 110 before impinging on the sensor array 120.

The color filter 110 may include a red filter region that includes a red color photoresist according to an embodiment. The red color photoresist may include a polymer having a backbone at least a portion of which corresponds to a (meth)acrylate that includes an anthraquinone moiety having a transmittance spectrum producing red light, and a linking group covalently coupled to the anthraquinone moiety and an ester oxygen of the (meth)acrylate. The linking group may include phenyl, naphthyl, a linear alkyl group having from 2 to about 10 carbons, a branched alkyl group having from 3 to about 10 carbons, a cycloalkyl group having from about 3 to about 20 carbons, or a substituted aromatic group. In another implementation, the linking group may include phenyl, naphthyl, a linear alkyl group having from 3 to about 10 carbons, a branched alkyl group having from 4 to about 10 carbons, a cycloalkyl group having from about 3 to about 20 carbons, or a substituted aromatic group.

As described above, embodiments may provide materials suitable for a red color photoresist that exhibits thermal stability, light stability, and chemical stability. The red color photoresist may be used to fabricate, e.g., a color filter for a camera, the color filter being disposed adjacent to a sensor array such as a CMOS sensor. The red color photoresist may be suitable for the fabrication of pixels having a dimension smaller than that practicable with conventional pigment-based materials. For example, the red color photoresist according to embodiments may be used to fabricate pixels having a width of about 2 μm or less, e.g., 1.4 μm.

Example embodiments have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A (meth)acrylate ester, comprising:
a (meth)acrylate monomer moiety having an ester oxygen;
an anthraquinone moiety having a transmittance spectrum producing red light; and
a linking group covalently coupled to the ester oxygen and the anthraquinone moiety, wherein the linking group includes phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

2. The ester as claimed in claim 1, wherein the linking group is derived from an amino alcohol.

3. The ester as claimed in claim 1, wherein the anthraquinone moiety has the linking group at the 1-position only.

4. A method of synthesizing a (meth)acrylate ester, the method comprising:
providing an anthraquinone compound having a reactive group attached to a ring of the anthraquinone ring;
reacting a linking group with the reactive group such that the linking group becomes covalently bound to the ring of the anthraquinone; and
reacting a (meth)acrylic acid-derived compound with the linking group such that the linking group becomes covalently bound to an oxygen of the (meth)acrylic acid-derived compound, wherein:
the linking group includes phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

5. The method as claimed in claim 4, wherein the linking group is an amino alcohol.

6. The method as claimed in claim 4, wherein the anthraquinone moiety has the linking group at the 1-position only.

7. The method as claimed in claim 4, wherein the (meth)acrylic acid-derived compound is a (meth)acrylic acid anhydride.

8. A method of manufacturing a camera, the method comprising:
fabricating a color filter, the color filter including a red filter region formed therein; and
mounting the color filter proximate to a sensor array, wherein:
forming the red filter region includes patterning a red color photoresist, and
the red color photoresist includes a polymer having a backbone at least a portion of which corresponds to a (meth)acrylate that includes:
an anthraquinone moiety having a transmittance spectrum producing red light; and
a linking group covalently coupled to the anthraquinone moiety and an ester oxygen of the (meth)acrylate, the linking group including:
phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

9. The method as claimed in claim 8, wherein patterning the red color photoresist includes exposing the red color photoresist to i-line radiation.

10. The method as claimed in claim 8, wherein:
the red color photoresist is a negative resist,
the polymer includes at least one functional group that is polymerizable with another component of the red color photoresist by exposure to UV light, and
the polymer backbone includes at least one functional group that is reactive when placed in contact with aqueous alkaline developer.

11. The method as claimed in claim 8, wherein the red color photoresist includes:
the polymer,
a crosslinker, and
an initiator that is reactive to UV light.

12. The method as claimed in claim 11, wherein the crosslinker includes an acrylate oligomer.

13. The method as claimed in claim 12, wherein the acrylate oligomer includes a polyol (meth)acrylate ester.

14. The method as claimed in claim 8, wherein patterning the red color photoresist includes forming a red color pixel having a width of about 2 μm or less.

15. The method as claimed in claim 14, wherein patterning the red color photoresist includes forming a red color pixel having a width of about 1.4 μm or less.

16. A camera, comprising:
a sensor array; and
a color filter proximate to the sensor array, the color filter including a red filter region, wherein:
the red filter region includes a red color photoresist, and
the red color photoresist includes a polymer having a backbone at least a portion of which corresponds to a (meth)acrylate that includes:
an anthraquinone moiety having a transmittance spectrum producing red light; and
a linking group covalently coupled to the anthraquinone moiety and an ester oxygen of the (meth)acrylate, the linking group including:
phenyl, naphthyl, a linear alkyl group having from 2 to about 10 carbons, a branched alkyl group having from 3 to about 10 carbons, a cycloalkyl group having from about 3 to about 20 carbons, or a substituted aromatic group.

17. The camera as claimed in claim 16, wherein the red filter region includes a red color pixel having a width of about 2 μm or less.

18. The camera as claimed in claim 17, wherein the red filter region includes a red color pixel having a width of about 1.4 μm or less.
